(19) United States
(12) Patent Application Publication
(10) Pub. No.: US 2005/0096669 A1
(43) Pub. Date: May 5, 2005

(54) Apparatus and method for an ultrasonic medical device to treat coronary thrombus bearing lesions

(75) Inventors: Robert A. Rabiner, North Reading, MA (US); Bradley A. Hare, Chelmsford, MA (US); Craig T. Kelley, Lexington, MA (US)

Correspondence Address: PALMER & DODGE, LLP;RICHARD B. SMITH, 111 HUNTINGTON AVENUE, BOSTON, MA, 02199, US

(73) Assignee: OmniSonics Medical Technologies, Inc.

(21) Appl. No.: 10/976,268

(22) Filed: Oct. 28, 2004

Related U.S. Application Data
(63) Continuation-in-part of application No. 10/635,200, filed on Aug. 6, 2003, now Pat. No. 6,866,670.
Application No. 10/635,200 is a division of application No. 09/972,555, filed on Oct. 5, 2001, now Pat. No. 6,660,013.
Application No. 09/972,555 is a continuation-in-part of application No. 09/618,352, filed on Jul. 19, 2000, now Pat. No. 6,551,337.
(60) Provisional application No. 60/178,901, filed on Jan. 28, 2000.
(60) Provisional application No. 60/157,824, filed on Oct. 5, 1999.

(51) Int. Cl. A61B 17/22

(52) U.S. Cl. 606/128

(57) ABSTRACT

An apparatus and method for using an ultrasonic medical device to treat coronary thrombus bearing lesions comprises an ultrasonic probe, a transducer, a coupling engaging a proximal end of the ultrasonic probe to a distal end of the transducer and an ultrasonic energy source engaged to the transducer. The ultrasonic probe is inserted into a vasculature in communication with the coronary thrombus bearing lesion. The ultrasonic energy source produces energy that is transmitted to the transducer, which generates a transverse ultrasonic vibration along the ultrasonic probe. The transverse ultrasonic vibration creates a plurality of transverse nodes and a plurality of transverse anti-nodes along the longitudinal axis of the ultrasonic probe, creating cavitation along a portion of the longitudinal axis of the ultrasonic probe to ablate the coronary thrombus bearing lesion.

### RELATED APPLICATIONS

This is a continuation-in-part of application Ser. No. 10/635,200, filed Aug. 6, 2003, which is a divisional of application Ser. No. 09/972,555, filed Oct. 5, 2001, now U.S. Pat. No. 6,660,013, which is a continuation-in-part of application Ser. No. 09/618,352, filed Jul. 19, 2000, now U.S. Pat. No. 6,551,337, which claims benefit of Provisional Application Ser. No. 60/178,901, filed Jan. 28, 2000, and claims benefit of Provisional Application Ser. No. 60/157,824, filed Oct. 5, 1999, the entirety of all these applications are hereby incorporated herein by reference.

### FIELD OF THE INVENTION

The present invention relates to medical devices, and more particularly to an apparatus and a method for an ultrasonic medical device to treat coronary thrombus bearing lesions.

### BACKGROUND OF THE INVENTION

Coronary thrombus bearing lesions are a leading cause of death and impairment in the world today. The coronary thrombus bearing lesions result in different degrees of severity ranging from stable angina to acute coronary syndrome and ultimately to a heart attack. The coronary thrombus bearing lesions compromise the functionality of the heart and the circulatory system.

The cardiovascular system of the body includes the heart and the circulatory system, including the vasculatures carrying blood to and from the heart. The heart is the body's hardest working organ, and like all body organs, the heart requires a supply of blood to bring it oxygen. Throughout life, the heart continuously pumps blood enriched with oxygen and vital nutrients through a network of arteries to all parts of the body's tissues. Since blood passes through the heart so quickly and with a high pressure, the heart is unable to get oxygen from the blood within its chambers. In order to receive oxygen rich blood, the muscle comprising the wall of the heart, the myocardium, receives oxygen rich blood through a network of small arteries branching from the aorta. This network of small arteries, better known as the coronary arteries, cross over the surface of the heart where they divide and send tiny branches into the heart muscle. To function properly, the heart muscle must be provided oxygen rich blood and oxygen depleted blood must be carried away.

The coronary arteries consist of two main arteries, the right coronary artery and the left coronary artery. The left coronary artery supplies blood to the heart ventricles and the left atrium. The left coronary artery divides into the left anterior descending artery and the circumflex branch. The left anterior descending artery passes over the front of the heart toward the apex (tip) and supplies the front surface and tip of the heart and the front part of the septum, the wall between the right and left ventricles which are the main pumping chambers. The circumflex branch lies in a groove between the left atrium and the left ventricle and supplies the portion of the left ventricular wall away from the septum. The right coronary artery, which divides into the right posterior descending artery and a large marginal branch, supplies blood to the heart ventricles, right atrium and sino-atrial node, a cluster of cells in the right atrial wall that regulates the heart's pumping rhythm. From the large coronary vessels, smaller branches arise, which divide and insert into the heart muscle, supplying its nutritional needs.

There are three basic ways to treat atherosclerotic disease: medication, surgery, and minimally invasive interventional procedures such as stent implantation, percutaneous transluminal coronary angioplasty (PTCA), intravascular radiotherapy, atherectomy, and excimer laser therapy. A combination of these therapies may be used to treat atherosclerotic disease. The purpose of these treatments is to eliminate or reduce the symptoms and, in the case of coronary artery disease, decrease the risk of heart attack.

An angioplasty opens blocked arteries and allows blood to flow to the heart muscle. Angioplasty is not a surgical procedure, requires only local anesthetic, and generally takes thirty minutes to an hour for the procedure to be completed. The process of unblocking the artery requires the insertion of a small tube (guide catheter) into an artery in the groin or arm. The tube is fed through the arterial system until it reaches the blocked coronary artery. A thin expendable balloon is inserted through the tube and inflated inside the clogged artery so that it pushes the obstruction to the side and clears open a path for the blood to flow more easily.

Coronary thrombosis, also known as a myocardial infarction or heart attack, usually takes place in the coronary arteries, and frequently develops at the site of an atherosclerotic plaque rupture. If a blood clot develops in one of these arteries, the blood supply to that area of the heart muscle will be reduced or stop. The area of muscle to which there is insufficient blood supply stops working properly if the blood clot is not dissolved quickly, e.g. with thrombosis dissolving (thrombolytic) medication.

Medications can be used alone or in combination with one of the treatments. While medications do not eliminate the narrowing of arteries, they can remove thrombus and help improve the efficiency of the heart and reduce symptoms such as chest pain (angina), leg pain, claudication, and hypertension.

Coronary thrombus bearing lesions are a primary cause of coronary artery disease. U.S. Pat. No. 6,262,062 to Clemens, U.S. Pat. No. 6,258,798 to Wallentin, U.S. Pat. No. 6,440,947 to Barron et al. and U.S. Pat. No. 6,451,303 to Whitehouse et al. disclose the use of various drugs to treat coronary artery disease. The use of drugs to treat coronary artery disease does not completely remove the thrombus, and the particulate that is created by breaking down the clot is carried downstream where it can cause a secondary thrombus downstream. In addition, the use of drugs to treat coronary artery disease can have detrimental side effects and pose health risks to the patient.

The prior art does not provide a solution for effectively treating coronary artery disease and coronary thrombus bearing lesions. The prior art does not remove the thrombus comprising the coronary artery disease and the prior art presents adverse consequences to the patient. Therefore, there remains a need in the art for an apparatus and a method of preventing and treating coronary artery disease that effectively removes coronary thrombus bearing lesions in a safe, effective and time efficient manner.

### SUMMARY OF THE INVENTION

An apparatus and method for using an ultrasonic medical device to treat a coronary thrombus bearing lesion comprises an ultrasonic probe, a transducer, a coupling engaging a proximal end of the ultrasonic probe to a distal end of the transducer and an ultrasonic energy source engaged to the transducer. The ultrasonic probe is inserted into a vasculature and placed in communication with the coronary thrombus bearing lesion. The ultrasonic energy source produces energy that is transmitted to the transducer, which generates a transverse ultrasonic vibration along the ultrasonic probe. The transverse ultrasonic vibration creates a plurality of transverse nodes and a plurality of transverse anti-nodes along the longitudinal axis of the ultrasonic probe, creating cavitation along a portion of the longitudinal axis of the ultrasonic probe to ablate the coronary thrombus bearing lesion.

An ultrasonic medical device for treating a coronary thrombus bearing lesion comprises an ultrasonic probe having a proximal end, a distal end and a longitudinal axis therebetween; a transducer creating a transverse ultrasonic vibration along at least a portion of the longitudinal axis of the ultrasonic probe; a coupling engaging the proximal end of the ultrasonic probe to a distal end of the transducer; and an ultrasonic energy source engaged to the transducer, wherein a transverse ultrasonic vibration generates a plurality of transverse nodes and a plurality of transverse anti-nodes along at least a portion of the longitudinal axis of the ultrasonic probe, creating cavitation in a medium surrounding the ultrasonic probe to treat the coronary thrombus bearing lesion.

An ultrasonic medical device for ablating a coronary thrombus bearing lesion comprises an ultrasonic probe having a proximal end, a distal end terminating in a probe tip and a longitudinal axis between the proximal end and the distal end; a transducer that converts electrical energy into mechanical energy, creating a transverse ultrasonic vibration along the longitudinal axis of the ultrasonic probe; and a coupling engaging the proximal end of the ultrasonic probe to a distal end of the transducer, wherein the transverse ultrasonic vibration produces a plurality of transverse nodes and a plurality of transverse anti-nodes along a portion of the longitudinal axis of the ultrasonic probe.

The present invention also provides a method of resolving a coronary thrombus bearing lesion comprising: providing an ultrasonic probe having a proximal end, a distal end and a longitudinal axis therebetween is provided; navigating the ultrasonic probe adjacent to the coronary thrombus bearing lesion; placing the ultrasonic probe in communication with the coronary thrombus bearing lesion; activating an ultrasonic energy source engaged to the ultrasonic probe to generate a transverse ultrasonic vibration along at least a portion of the longitudinal axis of the ultrasonic probe, wherein the transverse ultrasonic vibration creates a plurality of transverse nodes and a plurality of transverse anti-nodes along a portion of the longitudinal axis of the ultrasonic probe.

The present invention also provides a method of ablating a coronary thrombus bearing lesion in a coronary artery of a vasculature comprising: providing an ultrasonic medical device comprising an ultrasonic probe having a proximal end, a distal end terminating in a probe tip, and a longitudinal axis between the proximal end and the distal end; inserting the ultrasonic probe in an insertion point in the vasculature; moving the ultrasonic probe to place the ultrasonic probe in communication with the coronary thrombus bearing lesion in the coronary artery; activating an ultrasonic energy source engaged to the ultrasonic probe to produce an electric signal that drives a transducer of the ultrasonic medical device to produce a transverse ultrasonic vibration of the ultrasonic probe, wherein the transverse ultrasonic vibration produces cavitation in a medium surrounding the ultrasonic probe to ablate the coronary thrombus bearing lesion.

The present invention also provides a method of resolving a coronary thrombus bearing lesion comprising: providing an ultrasonic medical device comprising an ultrasonic probe having a proximal end, a distal end and a longitudinal axis therebetween, wherein the ultrasonic probe comprises at least one radiopaque marker; navigating the ultrasonic probe adjacent to the coronary thrombus bearing lesion; viewing the ultrasonic probe using a fluoroscopic procedure; placing the ultrasonic probe in communication with the coronary thrombus bearing lesion; activating an ultrasonic energy source engaged to the ultrasonic probe to generate a transverse ultrasonic vibration along at least a portion of the longitudinal axis of the ultrasonic probe, wherein the transverse ultrasonic vibration creates a plurality of transverse nodes and a plurality of transverse anti-nodes along a portion of the longitudinal axis of the ultrasonic probe.

In an embodiment, the ultrasonic medical device includes at least one radiopaque marker located along the longitudinal axis of the ultrasonic probe. The radiopaque marker allows the ultrasonic probe to be visualized through a fluoroscopic procedure. In another embodiment, the ultrasonic probe contains a super-elastic alloy. In another embodiment, a segment of the ultrasonic probe is sheathed in a thin wall polymer hypotube for fluoroscopic visibility, tip softness, and/or efficient energy transmission.

The present invention provides an apparatus and a method for an ultrasonic medical device to treat a coronary thrombus bearing lesion. An ultrasonic probe is placed in communication with a coronary thrombus bearing lesion and a transverse ultrasonic vibration along at least a portion of the longitudinal axis of the ultrasonic probe ablates the coronary thrombus bearing lesion. The present invention provides an ultrasonic medical device for treating a coronary thrombus bearing lesion that is simple, user-friendly, time efficient, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a side plan view of an ultrasonic probe of the present invention inserted into a vasculature adjacent to a coronary thrombus bearing lesion in a heart.

FIG. 2 is a side plan view of an ultrasonic probe of the present invention capable of ablating a coronary thrombus bearing lesion.

FIG. 3 is a side plan view of an embodiment of an ultrasonic probe of the present invention capable of ablating a coronary thrombus bearing lesion where a diameter of the ultrasonic probe is approximately uniform from the proximal end of the ultrasonic probe to the distal end of the ultrasonic probe.

FIG. 4 is a side plan view of an ultrasonic probe of the present invention showing a plurality of transverse nodes and a plurality of transverse anti-nodes along a portion of a longitudinal axis of the ultrasonic probe for ablating a coronary thrombus bearing lesion.

FIG. 5 is an enlarged view of a heart showing the coronary arteries and coronary thrombus bearing lesion in a coronary artery.

FIG. 6 is an enlarged view of an ultrasonic probe of the present invention adjacent to the coronary thrombus bearing lesion in a coronary artery of a heart.

FIG. 7 is an enlarged view of an ultrasonic probe of the present invention showing a plurality of transverse nodes and a plurality of transverse anti-nodes in communication with a coronary thrombus bearing lesion.

FIG. 8 is a side plan view of an embodiment of an ultrasonic probe of the present invention capable of ablating a coronary thrombus bearing lesion where an intermediate material engages the ultrasonic probe and a flexible material extends from the intermediate material.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The following terms and definitions are used herein:

“Ablate” as used herein refers to removing, clearing, destroying or taking away a thrombus. “Ablation” as used herein refers to a removal, clearance, destruction, or taking away of the thrombus.

“Anti-node” as used herein refers to a region of a maximum energy emitted by an ultrasonic probe at or adjacent to a specific location along a longitudinal axis of the ultrasonic probe.

“Node” as used herein refers to a region of a minimum energy emitted by an ultrasonic probe at or adjacent to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe).

“Thrombus” as used herein refers to a collection of a matter including, but not limited to, a group of similar cells, intravascular blood clots, occlusions, plaque, biological material, fibrin, calcified plaque, calcium deposits, occlusional deposits, atherosclerotic plaque, fatty deposits, adipose tissues, atherosclerotic cholesterol buildup, fibrous material buildup, arterial stenoses, minerals, high water content tissues, platelets, cellular debris, wastes and other occlusive materials.

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Vasculature” as used herein refers to the entire circulatory system for the blood supply including the venous system, the arterial system and the associated vessels, arteries, veins, capillaries, blood, and the heart. The arterial system is the means by which blood with oxygen and nutrients is transported to tissues. The venous system is the means by which blood with carbon dioxide and metabolic by-products is transported for excretion.

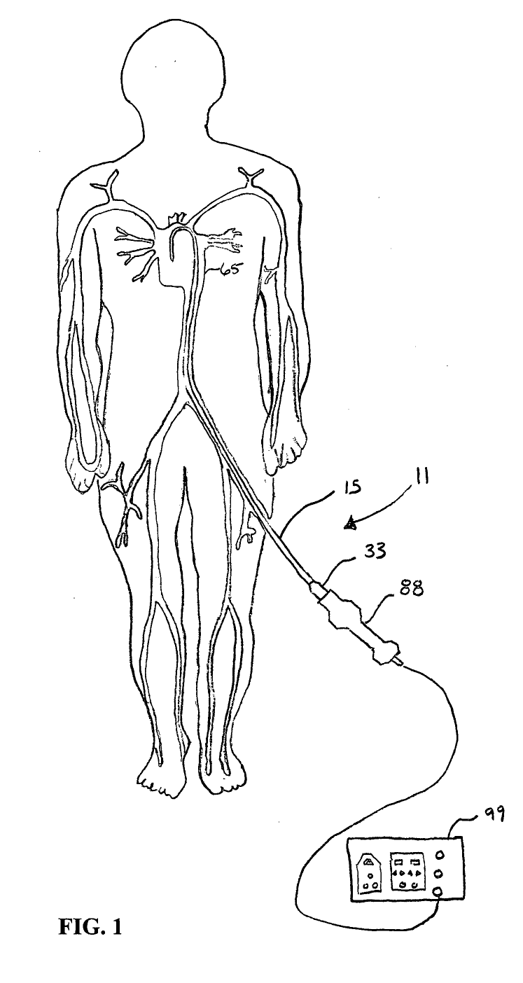

An ultrasonic medical device of the present invention capable of ablating a coronary thrombus bearing lesion is illustrated generally at 11 in FIG. 1. The ultrasonic medical device 11 includes an ultrasonic probe 15 which is coupled to an ultrasonic energy source or generator 99 for the production of an ultrasonic energy. A flexibility of the ultrasonic probe 15 allows the ultrasonic probe 15 to be bent, deflected and flexed through the vasculatures toward the coronary arteries without compromising the integrity of the vasculature or the ultrasonic probe 15.

Coronary thrombus bearing lesions can cause coronary artery disease. Coronary artery disease is the most common cause of heart attacks, or myocardial infarction or coronary thrombosis. Coronary artery disease results from a complex process called atherosclerosis, commonly known as hardening of the arteries, in which a thrombus causes a blockage of the arteries, ischemia, and prevents oxygen-rich blood from reaching the heart. Associated with atherosclerosis, atheroma is characterized by a degeneration of the interior of an artery by deposits within its wall and has the effect of narrowing the lumen (channel) of the artery, thus restricting blood flow. Atheroma predisposes to a number of conditions, including thrombosis, angina, and stroke.

There are several steps in the process leading to atherosclerosis with several theories to explain this process. Many scientists believe atherosclerosis begins when the innermost layer of the artery, the endothelium, becomes damaged. Atherosclerosis involves the slow buildup of deposits of fatty substances, cholesterol, body cellular waste products, calcium and fibrin in the inside lining of the artery. The resulting buildup, plaque, partially or totally blocks the flow of blood through the artery, leading to a formation of a blood clot, or thrombus, on the plaque's surface.

The severity of a resulting condition depends upon the amount of blockage in the vasculature. In a case of stable angina, the patient experiences chest pain and there is not believed to be significant blockage to the flow of blood through the artery. A case of acute coronary syndrome is a significant blockage of blood flow through the artery that has not developed into a full blown heart attack. Acute coronary syndromes include unstable angina, a serious situation that is an intermediate stage between stable angina and a heart attack, and non Q-wave myocardial infarction. A heart attack, more formally known as myocardial infarction, results when the flow of blood through the artery is blocked, causing tissue death from the lack of oxygen.

For coronary treatment procedures, time is of the essence. In cases of acute coronary ischemia and myocardial infarction, the health of the heart muscle supplied by the occluded artery will be largely determined by the time that elapses between the occlusive incident and the performance of the procedure. The use of a surface-active device of the present invention minimizes the need for interaction by the physician and will greatly reduce the treatment time, leading to better patient outcomes.

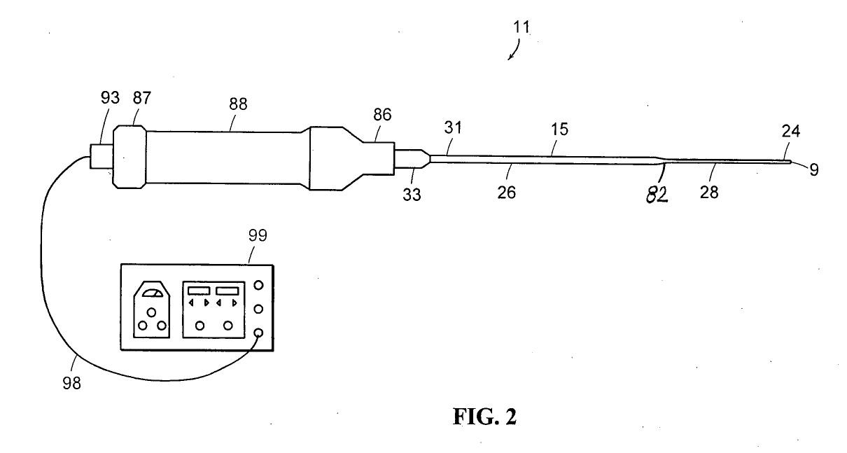

FIG. 2 shows the ultrasonic medical device 11 of the present invention. The ultrasonic probe 15 includes a proximal end 31, a distal end 24 that ends in a probe tip 9 and a longitudinal axis between the proximal end 31 and the distal end 24. A handle, 88, comprising a proximal end 87 and a distal end 86, surrounds a transducer within the handle 88. The transducer, having a proximal end engaging the ultrasonic energy source 99 and a distal end coupled to a proximal end 31 of the ultrasonic probe 15, transmits the ultrasonic energy to the ultrasonic probe 15. A connector 93 and a connecting wire 98 engage the ultrasonic energy source 99 to the transducer. In a preferred embodiment of the present invention shown in FIG. 2, a diameter of the ultrasonic probe 15 decreases from a first defined interval 26 to a second defined interval 28 along the longitudinal axis of the ultrasonic probe 15 over a transition 82. A coupling 33 that engages the proximal end 31 of the ultrasonic probe 15 to the transducer within the handle 88 is illustrated generally in FIG. 2. In a preferred embodiment of the present invention, the coupling 33 is a quick attachment-detachment system. An ultrasonic medical device with a rapid attachment and detachment means is described in the Assignee's U.S. Pat. No. 6,695,782 and Assignee's co-pending patent applications U.S. Ser. No. 10/268,487 and U.S. Ser. No. 10/268,843, which further describe the quick attachment-detachment system and the entirety of these patents and patent applications are hereby incorporated herein by reference.

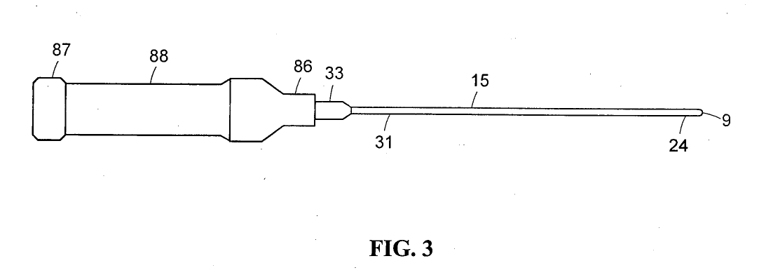

FIG. 3 shows an alternative embodiment of the ultrasonic probe 15 of the present invention. In the embodiment of the present invention shown in FIG. 3, the diameter of the ultrasonic probe 15 is approximately uniform from the proximal end 31 of the ultrasonic probe 15 to the distal end 24 of the ultrasonic probe 15.

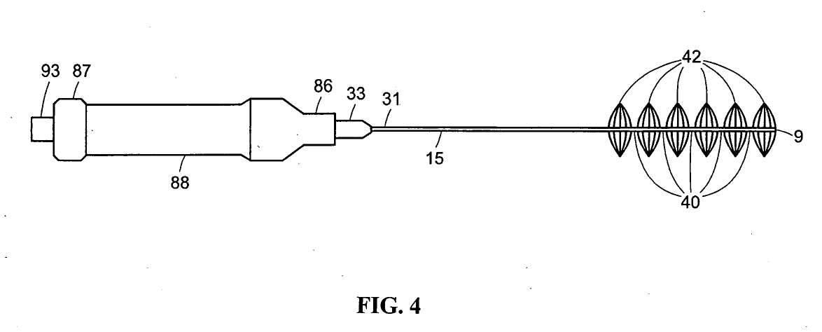

FIG. 4 shows a side plan view of an ultrasonic probe 15 of the present invention showing a plurality of transverse nodes 40 and a plurality of transverse anti-nodes 42 along a portion of a longitudinal axis of the ultrasonic probe 15.

In a preferred embodiment of the present invention, the ultrasonic probe 15 is a wire. In an embodiment of the present invention, the ultrasonic probe 15 is elongated. In an embodiment of the present invention, the diameter of the ultrasonic probe 15 changes at greater than two defined intervals. In an embodiment of the present invention, the transitions 82 of the ultrasonic probe 15 are tapered to gradually change the diameter from the proximal end 31 to the distal end 24 along the longitudinal axis of the ultrasonic probe 15. In another embodiment of the present invention, the transitions 82 of the ultrasonic probe 15 are stepwise to change the diameter from the proximal end 31 to the distal end 24 along the longitudinal axis of the ultrasonic probe 15. Those skilled in the art will recognize there can be any number of defined intervals and transitions, and the transitions can be of any shape known in the art and be within the spirit and scope of the present invention.

In an embodiment of the present invention, the gradual change of the diameter from the proximal end 31 to the distal end 24 occurs over the at least one transition 82, with each transition 82 having an approximately equal length. In another embodiment of the present invention, the gradual change of the diameter from the proximal end 31 to the distal end 24 occurs over a plurality of transitions 82 with each transition 82 having a varying length. The transition 82 refers to a section where the diameter varies from a first diameter to a second diameter.

In a preferred embodiment of the present invention, the ultrasonic probe 15 has a small diameter. In a preferred embodiment of the present invention, the cross section of the ultrasonic probe 15 is approximately circular. In another embodiment, the cross section of at least a portion of the ultrasonic probe 15 is non-circular. The ultrasonic probe 15 comprising a wire having a non-circular cross section at the distal end 24 can navigate through the vasculature. The ultrasonic probe 15 comprising a flat wire is steerable in the vasculature. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

In a preferred embodiment of the present invention, the diameter of the distal end 24 of the ultrasonic probe 15 is smaller than about 0.008 inches and the diameter of the proximal end 31 of the ultrasonic probe 15 is smaller than about 0.018 inches. Those skilled in the art will recognize the ultrasonic probe 15 can have a varying diameters at the proximal end 31 and the distal end 24 and be within the spirit and scope of the present invention.

The probe tip 9 can be any shape including, but not limited to, rounded, bent, a ball or larger shapes. In a preferred embodiment of the present invention, the probe tip 9 is smooth to prevent damage to the vasculature. In one embodiment of the present invention, the ultrasonic energy source 99 is a physical part of the ultrasonic medical device 11. In another embodiment of the present invention, the ultrasonic energy source 99 is not an integral part of the ultrasonic medical device 11. The ultrasonic probe 15 is used to ablate a thrombus and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

The ultrasonic probe 15 is designed, constructed and comprised of a material to not dampen the transverse ultrasonic vibration, and thereby supports a transverse vibration when flexed. In a preferred embodiment of the present invention, the ultrasonic probe 15 comprises titanium or a titanium alloy. Titanium is a strong, flexible, low density, low radiopacity and easily fabricated metal that is used as a structural material. Titanium and its alloys have excellent corrosion resistance in many environments and have good elevated temperature properties. In a preferred embodiment of the present invention, the ultrasonic probe 15 comprises titanium alloy Ti-6A1-4V. The elements comprising Ti-6A1-4V and the representative elemental weight percentages of Ti-6A1-4V are titanium (about 90%), aluminum (about 6%), vanadium (about 4%), iron (maximum about 0.25%) and oxygen (maximum about 0.2%). In another embodiment of the present invention, the ultrasonic probe 15 comprises stainless steel.

In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel.

In another embodiment of the present invention, the ultrasonic probe 15 comprises a super-elastic alloy. Even when bent or stretched, the super-elastic alloy returns to its original shape when the stress is removed. The ultrasonic probe 15 may contain super-elastic alloys known in the art including, but not limited to, nickel-titanium super-elastic alloys and Nitinol. Nitinol is a family of intermetallic materials, which contain a nearly equal mixture of nickel and titanium. Other elements can be added to adjust or tune the material properties. Nitinol is less stiff than titanium and is maneuverable in the vasculature. Nitonol has shape memory and super-elastic characteristics. The shape memory effect describes the process of restoring the original shape of a plastically deformed sample by heating it. This is a result of a crystalline phase change known as thermoelastic martensitic transformation. Below the transformation temperature, Nitinol is martensitic. Nitinol's excellent corrosion resistance, biocompatibility, and unique mechanical properties make it well suited for medical devices. Those skilled in the art will recognize that the ultrasonic probe 15 can be comprised of many other materials known in the art and be within the spirit and scope of the present invention.

The physical properties (i.e., length, cross sectional shape, dimensions, etc.) and material properties (i.e., yield strength, modulus, etc.) of the ultrasonic probe 15 are selected for operation of the ultrasonic probe 15 in the transverse mode. The length of the ultrasonic probe 15 of the present invention is chosen to be resonant in a transverse mode. In an embodiment of the present invention, the ultrasonic probe 15 is between about 30 centimeters and about 300 centimeters in length. In a preferred embodiment of the present invention, the length of the ultrasonic probe 15 is about 135 cm. Those skilled in the art will recognize an ultrasonic probe 15 can have a length shorter than about 30 centimeters, a length longer than about 300 centimeters and a length between about 30 centimeters and about 300 centimeters and be within the spirit and scope of the present invention.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer converts electrical energy provided by the ultrasonic energy source 99 to mechanical energy and sets the operating frequency of the ultrasonic medical device 11. The transducer is capable of engaging the ultrasonic probe 15 at the proximal end 31 with sufficient restraint to form an acoustical mass that can propagate the ultrasonic energy from the ultrasonic energy source 99.

In a preferred embodiment of the present invention, the ultrasonic probe 15 of the present invention is used to treat a coronary thrombus bearing lesion found within a coronary vasculature or a vasculature branching from the coronary vasculatures. In a preferred embodiment of the present invention, the ultrasonic medical device 11 of the present invention is used to treat coronary thrombus bearing lesions in the coronary arteries of the heart.

In addition to treating coronary thrombus bearing lesions, the ultrasonic medical device 11 of the present invention is used for deployment of other interventional devices including, but not limited to, catheters, guide catheters, sheaths, balloon catheters, stents and other interventional devices. In another embodiment of the present invention, the ultrasonic probe 15 of the ultrasonic medical device 11 is used as a coronary guidewire.

In a preferred embodiment of the present invention, the coronary arteries are accessed through a femoral puncture site. The femoral puncture creates an insertion point in a femoral artery in the leg. Those skilled in the art will recognize the coronary arteries can be accessed through puncture sites located at various locations within the body and be within the spirit and scope of the present invention.

In an embodiment of the present invention, an introducer is inserted through the femoral puncture and the ultrasonic probe 15 is moved within the introducer, through the vasculature, to the coronary artery and adjacent to the coronary thrombus bearing lesion 75. In another embodiment of the present invention, the ultrasonic probe 15 is inserted into the femoral artery and an introducer is moved over the ultrasonic probe 15. A device including, but not limited to, a vascular introducer can be used as the introducer to gain access to the femoral artery. A vascular introducer for use with an ultrasonic probe is described in Assignee's co-pending patent application U.S. Ser. No. 10/080,787, and the entirety of this application is hereby incorporated herein by reference. In another embodiment of the present invention a sheath is used to gain access to the femoral artery. In another embodiment of the present invention, a catheter is used to gain access to the femoral artery. In another embodiment of the present invention, access to the vasculature is gained through a peripheral artery.

In another embodiment of the present invention, the ultrasonic probe 15 of the present invention is advanced to the coronary thrombus bearing lesion 75 without using an introducer, sheath or catheter. Those skilled in the art will recognize the ultrasonic probe 15 can be advanced adjacent the coronary thrombus bearing lesion 75 in many ways known in the art and be within the spirit and scope of the present invention.

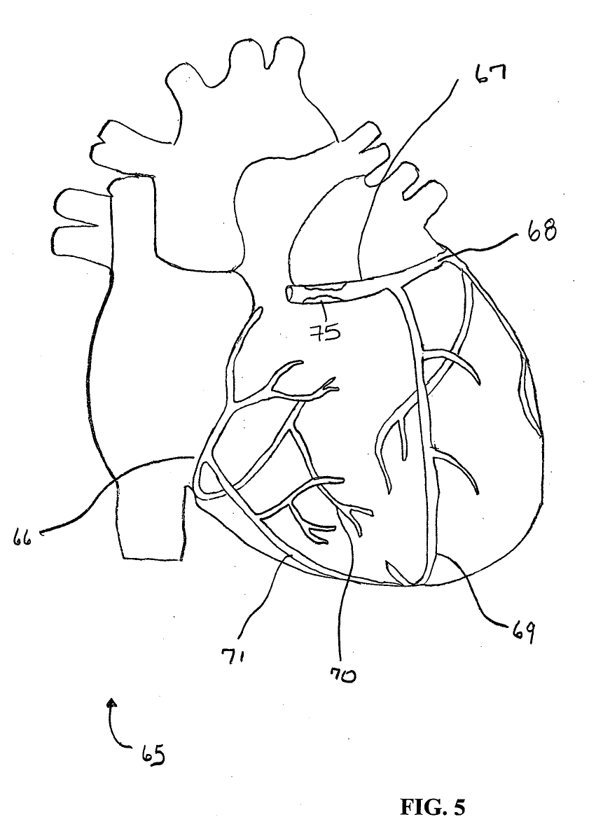

FIG. 5 shows a view of a heart 65 and coronary arteries of the heart 65. The coronary arteries supply blood to the heart muscle, which needs oxygen-rich blood to function while oxygen-depleted blood is carried away. The coronary arteries consist of two main arteries, the right coronary artery 66 and the left coronary artery 67. There are numerous branches from both the right coronary artery 66 and the left coronary artery 67. The left coronary artery 67, which supplies blood to the heart ventricles and left atrium, divides into a left anterior descending artery 69 and a circumflex branch 68. The right coronary artery 66, which supplies blood to the heart ventricles, right atrium, and sinostrial node, divides into a right posterior descending artery 70 and a large marginal branch 71. In FIG. 5, the left coronary artery 67 contains a coronary thrombus bearing lesion 75. The coronary thrombus bearing lesion 75 interrupts blood flow in the left coronary artery by the buildup or rupture of atheromous material within the left coronary artery 67, such that the blood flow to the left anterior descending artery 69 and the circumflex branch 68 are reduced.

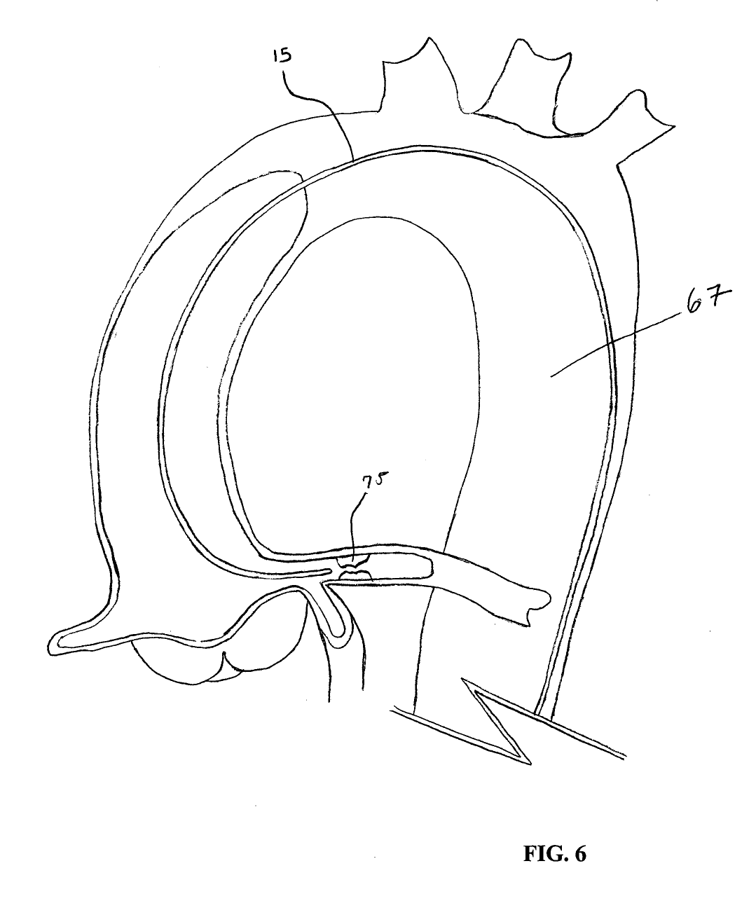

FIG. 6 shows an exploded view of a portion of the left coronary artery 67 with the ultrasonic probe 15 of the present invention adjacent to a coronary thrombus bearing lesion 75 within the left coronary artery 67. The coronary thrombus bearing lesion 75 interrupts blood flow in the left coronary artery by the buildup or rupture of atheromous material within the left coronary artery 67. The coronary thrombus bearing lesion 75 reduces a flow of oxygen and nutrients to the heart, which leads to complications including heart attacks and death.

The ultrasonic probe 15 is placed in communication with the coronary thrombus bearing lesion 75. In an embodiment of the present invention, the ultrasonic probe 15 is used to create a channel through the coronary thrombus bearing lesion 75. In another embodiment of the present invention, a pharmacological agent is injected into the coronary artery to soften the coronary thrombus bearing lesion 75. An apparatus and a method for an ultrasonic probe used with a pharmacological agent is described in Assignee's U.S. Pat. No. 6,733,451, and the entirety of this patent is hereby incorporated herein by reference.

After the ultrasonic probe 15 is placed in communication with the coronary thrombus bearing lesion 75, the ultrasonic energy source 99 is activated to provide a low power electric signal of between about 2 watts to about 15 watts to the transducer that is located within the handle 88. The transducer converts electrical energy provided by the ultrasonic energy source 99 to mechanical energy. The operating frequency of the ultrasonic medical device 11 is set by the transducer and the ultrasonic energy source 99 finds the resonant frequency of the transducer through a Phase Lock Loop. By an appropriately oriented and driven cylindrical array of piezoelectric crystals of the transducer, the horn creates a longitudinal wave along at least a portion of the longitudinal axis of the ultrasonic probe 15. The longitudinal wave is converted to a transverse wave along at least a portion of the longitudinal axis of the ultrasonic probe 15 through a nonlinear dynamic buckling of the ultrasonic probe 15.

As the transverse wave is transmitted along the longitudinal axis of the ultrasonic probe 15, a transverse ultrasonic vibration is created along the longitudinal axis of the ultrasonic probe 15. The ultrasonic probe 15 is vibrated in a transverse mode of vibration. The transverse mode of vibration of the ultrasonic probe 15 differs from an axial (or longitudinal) mode of vibration disclosed in the prior art. The transverse ultrasonic vibrations along the longitudinal axis of the ultrasonic probe 15 create a plurality of transverse nodes and a plurality of transverse anti-nodes along a portion of the longitudinal axis of the ultrasonic probe 15.

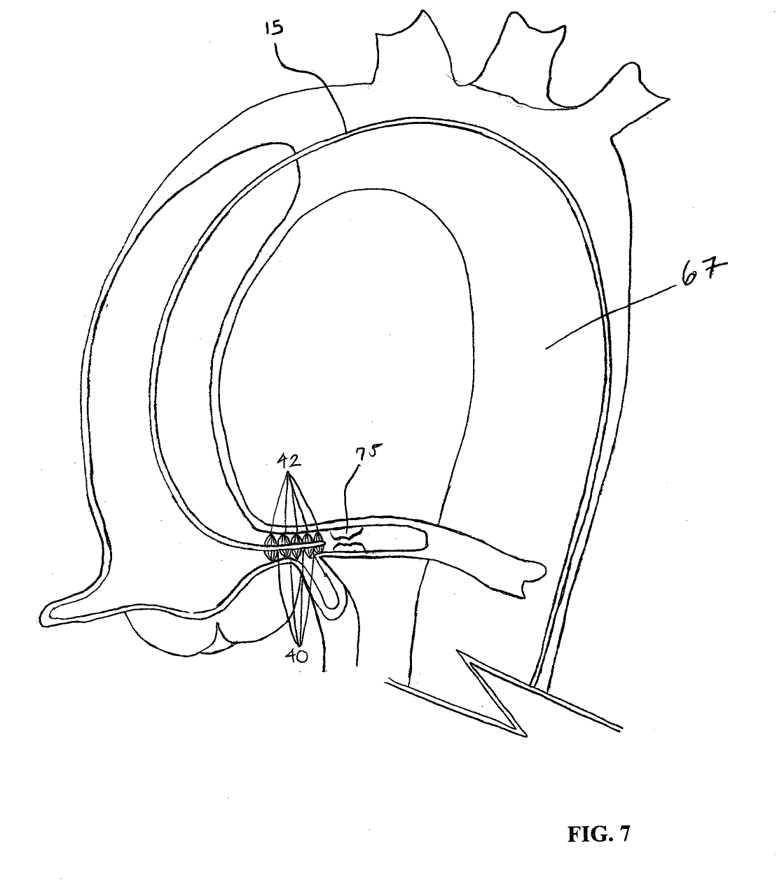

FIG. 7 shows the ultrasonic probe 15 of the present invention having a plurality of transverse nodes 40 and a plurality of transverse anti-nodes 42 along a portion of the longitudinal axis of the ultrasonic probe 15 and in communication with the coronary thrombus bearing lesion 75. The transverse nodes 40 are areas of minimum energy and minimum vibration that occur at repeating intervals along the portion of the longitudinal axis of the ultrasonic probe 15. The transverse anti-nodes 42, or areas of maximum energy and maximum vibration, also occur at repeating intervals along the portion of the longitudinal axis of the ultrasonic probe 15. The number of transverse nodes 40 and transverse anti-nodes 42, and the spacing of the transverse nodes 40 and transverse anti-nodes 42 of the ultrasonic probe 15 depend on the frequency of energy produced by the ultrasonic energy source 99. The separation of the transverse nodes 40 and transverse anti-nodes 42 is a function of the frequency, and can be affected by tuning the ultrasonic probe 15. In a properly tuned ultrasonic probe 15, the transverse anti-nodes 42 will be found at a position one-half of the distance between the transverse nodes 40 located adjacent to each side of the transverse anti-nodes 42.

The transverse wave is transmitted along the longitudinal axis of the ultrasonic probe 15 and the interaction of the surface of the ultrasonic probe 15 with the medium surrounding the ultrasonic probe 15 creates an acoustic wave in the surrounding medium. As the transverse wave is transmitted along the longitudinal axis of the ultrasonic probe 15, the ultrasonic probe 15 vibrates transversely. The transverse motion of the ultrasonic probe 15 produces cavitation in the medium surrounding the ultrasonic probe 15 to ablate the coronary thrombus bearing lesion 75. Cavitation is a process in which small voids are formed in a surrounding medium through the rapid motion of the ultrasonic probe 15 and the voids are subsequently forced to compress. The compression of the voids creates a wave of acoustic energy which acts to dissolve the matrix binding the coronary thrombus bearing lesion 75, while having no damaging effects on healthy tissue.

The coronary thrombus bearing lesion 75 in the coronary artery is resolved into a particulate having a size on the order of red blood cells (approximately 5 microns in diameter). The size of the particulate is such that the particulate is easily discharged from the body through conventional methods or simply dissolves into the blood stream. A conventional method of discharging the particulate from the body includes transferring the particulate through the blood stream to the kidney where the particulate is excreted as bodily waste.

The transverse ultrasonic vibration of the ultrasonic probe 15 results in a portion of the longitudinal axis of the ultrasonic probe 15 vibrated in a direction not parallel to the longitudinal axis of the ultrasonic probe 15. The transverse vibration results in movement of the longitudinal axis of the ultrasonic probe 15 in a direction approximately perpendicular to the longitudinal axis of the ultrasonic probe 15. Transversely vibrating ultrasonic probes for biological material ablation are described in the Assignee's U.S. Pat. No. 6,551,337; U.S. Pat. No. 6,652,547; U.S. Pat. No. 6,660,013; and U.S. Pat. No. 6,695,781, which further describe the design parameters for such an ultrasonic probe and its use in ultrasonic devices for ablation, and the entirety of these patents are hereby incorporated herein by reference.

As a consequence of the transverse ultrasonic vibration of the ultrasonic probe 15, the coronary thrombus destroying effects of the ultrasonic medical device 11 are not limited to those regions of the ultrasonic probe 15 that may come into contact with the coronary thrombus bearing lesion 75. Rather, as a section of the longitudinal axis of the ultrasonic probe 15 is positioned in proximity to the coronary thrombus bearing lesion 75, the coronary thrombus bearing lesion 75 is removed in all areas adjacent to the plurality of energetic transverse anti-nodes 42 that are produced along the portion of the length of the longitudinal axis of the ultrasonic probe 15, typically in a region having a radius of up to about 6 mm around the ultrasonic probe 15.

A novel feature of the present invention is the ability to utilize ultrasonic probes 15 of extremely small diameter compared to prior art probes, without loss of efficiency, because the coronary thrombus fragmentation process is not dependent on the area of the probe tip 9. Highly flexible ultrasonic probes 15 can therefore be designed for facile insertion into coronary thrombus bearing lesion areas or extremely narrow interstices that contain the coronary thrombus bearing lesion 75. Another advantage provided by the present invention is the ability to rapidly move the coronary thrombus bearing lesion 75 from large areas within cylindrical or tubular surfaces.

The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of coronary thrombus bearing lesion destroying transverse anti-nodes 42 along the longitudinal axis of the ultrasonic probe 15. The low frequency requirement of the present invention is a further advantage in that the low frequency requirement leads to less damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to the coronary thrombus bearing lesion 75 selectively, because the ultrasonic probe 15 conducts energy across a frequency range from about 10 kHz through about 100 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 10 kHz to about 100 kHz. In a preferred embodiment of the present invention, the frequency of ultrasonic energy is from about 20 kHz to about 40 kHz.

In an embodiment, a segment of the ultrasonic probe 15 is sheathed in a thin wall polymer hypotube for fluoroscopic visibility, tip softness, and/or efficient energy transmission.

In an embodiment of the present invention, the ultrasonic probe 15 comprises at least one material of high radiopacity that acts as a radiopaque marker. The material of high radiopacity does not allow the passage of a substantial amount of x-rays or other radiation and therefore allows a higher degree of visibility in an imaging procedure. The material of high radiopacity is biocompatible and non-toxic and is selected from a group including, but not limited to, tantalum, tungsten, gold, molybdenum, platinum, Nitinol, and alloys thereof. The material of high radiopacity could also be a polymer coated material. The material of high radiopacity is used as a radiopaque marker to enable a user to determine the length of a treatment zone. The material of high radiopacity may be located anywhere along the longitudinal axis of the ultrasonic probe 15. In one embodiment, two markers composed of materials of high radiopacity are located along the longitudinal axis of the ultrasonic probe 15. For example, one marker can be in the form of a band that is located on a polymeric sleeve, i.e., polytetrafluoroethylene (PTFE) or fluorinated ethylene propylene (FEP), surrounding the ultrasonic probe 15 and acts to mark the proximal end of the treatment zone and the other marker is located at the distal end 24 of the ultrasonic probe 15 to mark the distal end of the treatment zone. The material of high radiopacity at the distal end 24 of the ultrasonic probe 15 may be located at the probe tip 9. Under fluoroscopy, a treatment zone of the ultrasonic energy can be seen by the radiopaque markers on the ultrasonic probe 15. An ultrasonic medical device comprising a material of high radiopacity is described in Assignee's U.S. Pat. No. 6,730,048 and an ultrasonic medical device comprising a radiopaque marker is described in Assignee's co-pending patent application U.S. Ser. No. 10/207,468 (published patent application No. 2004/0019266), and the entirety of these patents and patent applications are hereby incorporated herein by reference.

In another embodiment of the present invention, a flexible material surrounds at least a portion of the longitudinal axis of the ultrasonic probe 15. The portion of the longitudinal axis of the ultrasonic probe 15 with the flexible material may be shaped to increase a radial span of the ultrasonic medical device 11. The flexible material protects the vasculature as the ultrasonic probe 15 is moved through the vasculature. In another embodiment of the present invention, the flexible material may extend beyond the probe tip 9. The flexible material may comprise a material of high radiopacity to enhance the visibility of the ultrasonic medical device 11 during certain medical procedures. An ultrasonic medical device engaging a flexible material is described in Assignee's co-pending patent application U.S. Ser. No. 10/646,408, and the entirety of this patent application is hereby incorporated herein by reference.

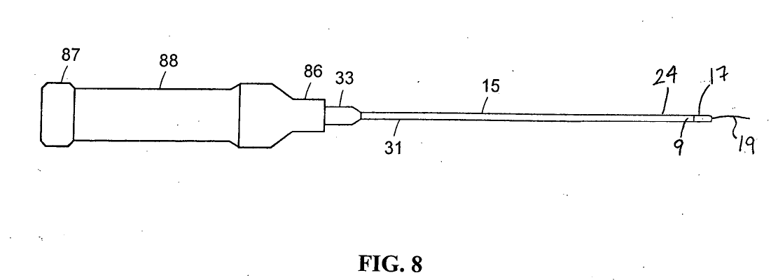

FIG. 8 shows an embodiment of the ultrasonic probe 15 of the present invention in which a flexible material 19 extends from the distal end 24 of the ultrasonic probe 15. The flexible material 19 engages the ultrasonic probe 15 by an intermediate material 17. The intermediate material 17 may be a dense material with high radiopacity that engages the probe tip 9. The intermediate material 17 engages the probe 15 through processes including, but not limited to, mechanically engaging and metallurgically engaging. The more specific processes of engaging two materials include, but are not limited to, welding, brazing, shrink fitting, lap welding, threaded fitting, butt-welding, twisting the materials and other mechanical or metallurgical connections. Those skilled in the art will recognize that other processes known in the art for engaging the intermediate material and the ultrasonic probe would be within the spirit and scope of the present invention.

The flexible material 19 can be a radiopaque material that enables the ultrasonic probe 15 to be visualized through a fluoroscopic procedure. Use of a radiopaque material for the flexible material 19 enhances the utility of the ultrasonic probe 15 of the present invention, as longer radiopaque sections are easier to view through fluoroscopy or other imaging procedures. If the flexible material 19 is sufficiently dense, the transverse wave will reflect off the proximal portion of the flexible material 19 and only a minimal amount of ultrasonic vibration will be transmitted beyond the probe tip 9 of the ultrasonic probe 15. As the material stress is proportional to the amplitude of the ultrasonic vibration, minimizing the transmitted ultrasonic vibration will allow materials with low mechanical strength to be joined to the vibrating ultrasonic probe 15. The fraction of the wave reflected from the intermediate material 17 will depend on the characteristic mechanical impedance of the materials given by the equation:
Z₀=ρc
Where Z₀ is the mechanical impedance, ρ is the density and c is the speed of sound. With known characteristic impedances, the reflection coefficient, or the fraction reflected, for longitudinal and torsion modes will be:

$$Γ = \frac{{Z}_{I} - 1}{{Z}_{W} + 1}$$

Where Γ is the reflection coefficient, Z_{I} is the characteristic impedance of the intermediate material 17 and Z_{W} the characteristic impedance of the ultrasonic probe 15. For transverse waves, the calculation of the reflection coefficient is more complex due to the inherent dispersion (variation of the speed of sound with frequency) and due to the fact that both moments and forces must be balanced at the interfaces between the three segments (i.e., the distal end of the probe 24, the intermediate material 17 and the flexible material 19). For transverse wave propagation, the reflection coefficients are best calculated from finite element models of the desired structure. The value of the reflection coefficient chosen will depend on the mechanical strength of the flexible material 19 attached to the intermediate material 17. As an example, for a material with about one-half the mechanical strength of titanium the allowable vibration amplitude on the flexible material 19 would need to be reduced by about one-half.

In one embodiment of the present invention, the intermediate material 17 is a dense material with high radiopacity that engages the distal end 24 of the ultrasonic probe 15. In an embodiment of the invention, tantalum is used as the intermediate material 17 to join together a titanium ultrasonic probe 15 with a flexible radiopaque material such as platinum, Nitinol or a polymer coated material. Using a radiopaque material for the flexible section greatly enhances the utility of the device, as longer radiopaque sections are easier to view through fluoroscopy or other imaging procedures. The use of an intermediate material 17 also allows the attachment by welding of materials that would not be compatible with titanium, but would be compatible with the intermediate material. The use of a flexible material 19 that extends from the probe tip 9 enables navigation within a vessel.

As discussed above, the ultrasonic medical device 11 of the present invention is used for deployment of other interventional devices. In an embodiment, a guide wire is used to position a guide catheter at either a right or left coronary ostium. The ultrasonic probe 15 of the present invention is then passed through the guide catheter and moves past a distal end of the guide catheter. The guide catheter allows deployment of various treatment and diagnostic devices. An apparatus and method for an ultrasonic medical device having a probe with a small proximal end for permitting over the probe transfers of vascular intervention devices is described in Assignee's co-pending patent application U.S. Ser. No. 10/959,703, and the entirety of this patent application is hereby incorporated herein by reference.

In an embodiment of the present invention, the ultrasonic probe 15 is passed through the guide catheter and moved past a distal end of the guide catheter to an area adjacent to the coronary thrombus bearing lesion 75 where it is used for ablation of the coronary thrombus bearing lesion 75. A balloon and/or a stent is then guided over the ultrasonic probe 15 to the coronary thrombus bearing lesion 75 site. In another embodiment of the present invention, the ultrasonic probe 15 is removed after treating the coronary thrombus bearing lesion 75 and replaced with a conventional guide wire, which is then used to guide the balloon and/or stent to the site of the coronary thrombus bearing lesion 75. In another embodiment of the invention, the ultrasonic probe 15 and the balloon and/or stent are deployed simultaneously within the guide catheter.

The ultrasonic probe 15 is placed in communication with the coronary thrombus bearing lesion 75 by moving, sweeping, bending, twisting or rotating the ultrasonic probe 15 along the coronary thrombus bearing lesion 75. Those skilled in the art will recognize that the many ways to move the ultrasonic probe in communication with the coronary thrombus bearing lesion known in the art are within the spirit and scope of the present invention.

The present invention also is a method of resolving a coronary thrombus bearing lesion 75. Access to the vasculature is gained by creating an insertion point in the vasculature using a device such as a vascular introducer. The ultrasonic probe 15 having a proximal end 31, a distal end 24 and a longitudinal axis therebetween is inserted through the insertion point of the vasculature and navigated through the vasculature and placed in communication with the coronary thrombus bearing lesion 75. A stiffness of the ultrasonic probe 15 of the ultrasonic medical device 11 gives the ultrasonic probe 15 a flexibility allowing the ultrasonic probe 15 to be deflected, flexed and bent through the tortuous paths of the vasculature, including the right coronary artery 66 and the left coronary artery 67. The ultrasonic energy source 99 engaged to the ultrasonic probe 15 is activated to generate an electric signal to drive the transducer of the ultrasonic medical device 11 to produce a transverse vibration of the ultrasonic probe 15. The transverse ultrasonic vibration of the ultrasonic probe 15 creates a plurality of transverse nodes 40 and a plurality of transverse anti-nodes 42 along a portion of the longitudinal axis of the ultrasonic probe 15 to resolve the coronary thrombus bearing lesion 75.

The present invention also provides a method of ablating a coronary thrombus bearing lesion 75 in a coronary artery of a vasculature comprising: providing an ultrasonic medical device 11 comprising an ultrasonic probe 15 having a proximal end 31, a distal end 24 terminating in a probe tip 9, and a longitudinal axis between the proximal end 31 and the distal end 24; inserting the ultrasonic probe 15 in an insertion point in the vasculature; moving the ultrasonic probe 15 to place the ultrasonic probe 15 in communication with the coronary thrombus bearing lesion 75 in the coronary artery; and activating an ultrasonic energy source 99 engaged to the ultrasonic probe 15 to produce an electric signal that drives a transducer of the ultrasonic medical device 11 to produce a transverse ultrasonic vibration of the ultrasonic probe 15, wherein the transverse ultrasonic vibration produces cavitation in a medium surrounding the ultrasonic probe 15 to ablate the coronary thrombus bearing lesion 75.

The present invention also provides a method of resolving a coronary thrombus bearing lesion 75 comprising: providing an ultrasonic medical device 11 comprising an ultrasonic probe 15 having a proximal end 31, a distal end 24 and a longitudinal axis therebetween, wherein the ultrasonic probe 15 comprises at least one radiopaque marker; navigating the ultrasonic probe 15 adjacent to the coronary thrombus bearing lesion 75; viewing the ultrasonic probe 15 using a fluoroscopic procedure; placing the ultrasonic probe 15 in communication with the coronary thrombus bearing lesion 75; and activating an ultrasonic energy source 99 engaged to the ultrasonic probe 15 to generate a transverse ultrasonic vibration along at least a portion of the longitudinal axis of the ultrasonic probe 15, wherein the transverse ultrasonic vibration creates a plurality of transverse nodes 40 and a plurality of transverse anti-nodes 42 along a portion of the longitudinal axis of the ultrasonic probe 15.

In an alternative embodiment of the present invention, the ultrasonic probe 15 is vibrated in a torsional mode. In the torsional mode of vibration, a portion of the longitudinal axis of the ultrasonic probe 15 comprises a radially asymmetric cross section and the length of the ultrasonic probe 15 is chosen to be resonant in the torsional mode. In the torsional mode of vibration, a transducer transmits ultrasonic energy received from the ultrasonic energy source 99 to the ultrasonic probe 15, causing the ultrasonic probe 15 to vibrate torsionally. The ultrasonic energy source 99 produces the electrical energy that is used to produce a torsional vibration along the longitudinal axis of the ultrasonic probe 15. The torsional vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of a plurality of torsional nodes and a section distal to each of the plurality of torsional nodes are vibrated out of phase, with the proximal section vibrated in a clockwise direction and the distal section vibrated in a counterclockwise direction, or vice versa. The torsional vibration results in an ultrasonic energy transfer to the biological material with minimal loss of ultrasonic energy that could limit the effectiveness of the ultrasonic medical device 11. The torsional vibration produces a rotation and a counterrotation along the longitudinal axis of the ultrasonic probe 15 that creates the plurality of torsional nodes and a plurality of torsional anti-nodes along a portion of the longitudinal axis of the ultrasonic probe 15 resulting in cavitation along the portion of the longitudinal axis of the ultrasonic probe 15 comprising the radially asymmetric cross section in a medium surrounding the ultrasonic probe 15 that ablates the biological material. An apparatus and method for an ultrasonic medical device operating in a torsional mode is described in Assignee's co-pending patent application U.S. Ser. No. 10/774,985, and the entirety of this application is hereby incorporated herein by reference.

In another embodiment of the present invention, the ultrasonic probe 15 is vibrated in a torsional mode and a transverse mode. A transducer transmits ultrasonic energy from the ultrasonic energy source 99 to the ultrasonic probe 15, creating a torsional vibration of the ultrasonic probe 15. The torsional vibration induces a transverse vibration along an active area of the ultrasonic probe 15, creating a plurality of nodes and a plurality of anti-nodes along the active area that result in cavitation in a medium surrounding the ultrasonic probe 15. The active area of the ultrasonic probe 15 undergoes both the torsional vibration and the transverse vibration.

Depending upon physical properties (i.e., length, diameter, etc.) and material properties (i.e., yield strength, modulus, etc.) of the ultrasonic probe 15, the transverse vibration is excited by the torsional vibration. Coupling of the torsional mode of vibration and the transverse mode of vibration is possible because of common shear components for the elastic forces. The transverse vibration is induced when the frequency of the transducer is close to a transverse resonant frequency of the ultrasonic probe 15. The combination of the torsional mode of vibration and the transverse mode of vibration is possible because for each torsional mode of vibration, there are many close transverse modes of vibration. By applying tension on the ultrasonic probe 15, for example by bending the ultrasonic probe 15, the transverse vibration is tuned into coincidence with the torsional vibration. The bending causes a shift in frequency due to changes in tension. In the torsional mode of vibration and the transverse mode of vibration, the active area of the ultrasonic probe 15 is vibrated in a direction not parallel to the longitudinal axis of the ultrasonic probe 15 while equally spaced points along the longitudinal axis of the ultrasonic probe 15 vibrate back and forth in a short arc about the longitudinal axis of the ultrasonic probe 15. An apparatus and method for an ultrasonic medical device operating in a transverse mode and a torsional mode is described in Assignee's co-pending patent application U.S. Ser. No. 10/774,898, and the entirety of this application is hereby incorporated herein by reference.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An ultrasonic medical device for treating a coronary thrombus bearing lesion comprising:
an ultrasonic probe having a proximal end, a distal end and a longitudinal axis therebetween;
a transducer creating a transverse ultrasonic vibration along at least a portion of the longitudinal axis of the ultrasonic probe;
a coupling engaging the proximal end of the ultrasonic probe to a distal end of the transducer; and
an ultrasonic energy source engaged to the transducer,
wherein the transverse ultrasonic vibration generates a plurality of transverse nodes and a plurality of transverse anti-nodes along at least a portion of the longitudinal axis of the ultrasonic probe, creating cavitation in a medium surrounding the ultrasonic probe to treat the coronary thrombus bearing lesion.

2. The ultrasonic medical device of claim 1 wherein the ultrasonic probe comprises a material that allows the ultrasonic probe to be bent, deflected and flexed.

3. The ultrasonic medical device of claim 1 wherein the ultrasonic probe comprises a diameter that enables insertion into a coronary artery.

4. The ultrasonic medical device of claim 1 wherein a diameter of the ultrasonic probe has a uniform diameter from the proximal end to the distal end.

5. The ultrasonic medical device of claim 1 wherein a diameter of the ultrasonic probe varies from the proximal end to the distal end.

6. The ultrasonic medical device of claim 1 wherein a cross section of the ultrasonic probe is approximately circular.

7. The ultrasonic medical device of claim 1 wherein the transverse ultrasonic vibration generates acoustic energy in a medium surrounding the ultrasonic probe.

8. The ultrasonic medical device of claim 1 wherein the ultrasonic energy source delivers energy in a frequency range from about 10 kHz to about 100 kHz.

9. The ultrasonic medical device of claim 1 wherein the ultrasonic energy source provides an electrical energy to the transducer at a resonant frequency of the transducer by finding the resonant frequency of the transducer.

10. The ultrasonic medical device of claim 1 wherein the ultrasonic probe is disposable.

11. The ultrasonic medical device of claim 1 further comprising at least one radiopaque marker located along the longitudinal axis of the ultrasonic probe.

12. The ultrasonic medical device of claim 11 wherein the radiopaque marker allows the ultrasonic probe to be visualized through a fluoroscopic procedure.

13. The ultrasonic medical device of claim 1 wherein the ultrasonic probe contains a super-elastic alloy.

14. An ultrasonic medical device for ablating a coronary thrombus bearing lesion comprising:
an ultrasonic probe having a proximal end, a distal end terminating in a probe tip and a longitudinal axis between the proximal end and the distal end;
a transducer that converts electrical energy into mechanical energy, creating a transverse ultrasonic vibration along the longitudinal axis of the ultrasonic probe; and
a coupling engaging the proximal end of the ultrasonic probe to a distal end of the transducer,
wherein the transverse ultrasonic vibration produces a plurality of transverse nodes and a plurality of transverse anti-nodes along a portion of the longitudinal axis of the ultrasonic probe.

15. The ultrasonic medical device of claim 14 wherein the ultrasonic probe supports the transverse ultrasonic vibration when flexed.

16. The ultrasonic medical device of claim 14 wherein the ultrasonic probe has a flexibility allowing the ultrasonic probe to be deflected and articulated.

17. The ultrasonic medical device of claim 14 wherein the transverse ultrasonic vibration along the longitudinal axis of the ultrasonic probe interacts with a medium surrounding the ultrasonic probe to create an acoustic wave in the medium.

18. The ultrasonic medical device of claim 14 wherein the transverse ultrasonic vibration of the ultrasonic probe produces cavitation in a medium surrounding the ultrasonic probe to ablate the coronary thrombus bearing lesion.

19. The ultrasonic medical device of claim 14 wherein an ultrasonic energy source engages the transducer to provide the electrical energy to the transducer.

20. The ultrasonic medical device of claim 14 further comprising at least one radiopaque marker located along the longitudinal axis of the ultrasonic probe.

21. The ultrasonic medical device of claim 20 wherein the radiopaque marker allows the ultrasonic probe to be visualized through a fluoroscopic procedure.

22. The ultrasonic medical device of claim 14 wherein the ultrasonic probe contains a super-elastic alloy.

23. A method of resolving a coronary thrombus bearing lesion comprising:
providing an ultrasonic medical device comprising an ultrasonic probe having a proximal end, a distal end and a longitudinal axis therebetween;
navigating the ultrasonic probe adjacent to the coronary thrombus bearing lesion;
placing the ultrasonic probe in communication with the coronary thrombus bearing lesion; and
activating an ultrasonic energy source engaged to the ultrasonic probe to generate a transverse ultrasonic vibration along at least a portion of the longitudinal axis of the ultrasonic probe,
wherein the transverse ultrasonic vibration creates a plurality of transverse nodes and a plurality of transverse anti-nodes along a portion of the longitudinal axis of the ultrasonic probe.

24. The method of claim 23 further comprising generating acoustic energy in a medium surrounding the ultrasonic probe through the transverse ultrasonic vibration of the ultrasonic probe.

25. The method of claim 23 further comprising sweeping the ultrasonic probe along the coronary thrombus bearing lesion.

26. The method of claim 23 further comprising moving the ultrasonic probe back and forth along the coronary thrombus bearing lesion.

27. The method of claim 23 further comprising rotating the ultrasonic probe along the coronary thrombus bearing lesion.

28. The method of claim 23 further comprising providing an electrical energy to a transducer at a resonant frequency of the transducer by the ultrasonic energy source determining the resonant frequency of the transducer.

29. The method of claim 23 further comprising delivering ultrasonic energy in a frequency range from about 10 kHz to about 100 kHz by the ultrasonic energy source.

30. The method of claim 23 further comprising providing the ultrasonic probe having a flexibility allowing the ultrasonic probe to be deflected and articulated.

31. The method of claim 23 further comprising viewing a radiopaque marker on the ultrasonic probe using a fluoroscopic procedure.

32. The method of claim 23 wherein the ultrasonic probe contains a super-elastic alloy.

33. A method of ablating a coronary thrombus bearing lesion in a coronary artery of a vasculature comprising:
providing an ultrasonic medical device comprising an ultrasonic probe having a proximal end, a distal end terminating in a probe tip, and a longitudinal axis between the proximal end and the distal end;
inserting the ultrasonic probe in an insertion point in the vasculature;
moving the ultrasonic probe to place the ultrasonic probe in communication with the coronary thrombus bearing lesion in the coronary artery; and
activating an ultrasonic energy source engaged to the ultrasonic probe to produce an electric signal that drives a transducer of the ultrasonic medical device to produce a transverse ultrasonic vibration of the ultrasonic probe,
wherein the transverse ultrasonic vibration produces cavitation in a medium surrounding the ultrasonic probe to ablate the coronary thrombus bearing lesion.

34. The method of claim 33 further comprising transmitting a transverse wave from the transverse ultrasonic vibration along the longitudinal axis of the ultrasonic probe to create an acoustic wave in the medium surrounding the ultrasonic probe.

35. The method of claim 33 further comprising producing a plurality of transverse nodes and a plurality of transverse anti-nodes along a portion of the longitudinal axis of the ultrasonic probe by the transverse ultrasonic vibration.

36. The method of claim 35 wherein the plurality of transverse nodes are points of a minimum transverse ultrasonic vibration.

37. The method of claim 35 wherein the plurality of transverse anti-nodes are points of a maximum transverse ultrasonic vibration.

38. The method of claim 33 wherein the ultrasonic probe is for a single use on a single patient.

39. The method of claim 33 further comprising delivering ultrasonic energy in a frequency range of about 10 kHz to about 100 kHz by the ultrasonic energy source.

40. The method of claim 33 further comprising viewing a radiopaque marker on the ultrasonic probe using a fluoroscopic procedure.

41. The method of claim 33 wherein the ultrasonic probe contains a super-elastic alloy.

42. A method of resolving a coronary thrombus bearing lesion comprising:
providing an ultrasonic medical device comprising an ultrasonic probe having a proximal end, a distal end and a longitudinal axis therebetween, wherein the ultrasonic probe comprises at least one radiopaque marker;
navigating the ultrasonic probe adjacent to the coronary thrombus bearing lesion;
viewing the ultrasonic probe using a fluoroscopic procedure;
placing the ultrasonic probe in communication with the coronary thrombus bearing lesion; and
activating an ultrasonic energy source engaged to the ultrasonic probe to generate a transverse ultrasonic vibration along at least a portion of the longitudinal axis of the ultrasonic probe,
wherein the transverse ultrasonic vibration creates a plurality of transverse nodes and a plurality of transverse anti-nodes along a portion of the longitudinal axis of the ultrasonic probe.
